(19) United States
(12) Patent Application Publication
(10) Pub. No.: US 2020/0030054 A1
(43) Pub. Date: Jan. 30, 2020

(54) OBSERVATION SYSTEM FOR DENTAL AND MEDICAL TREATMENT

(71) Applicant: ALICE VISION CORPORATION, Tokyo (JP)

(72) Inventor: Junya OKAWARA, Tokyo (JP)

(21) Appl. No.: 16/448,246

(22) Filed: Jun. 21, 2019

(30) Foreign Application Priority Data
Jul. 30, 2018 (JP) 2018-141939

(51) Int. Cl. A61B 90/00; H05K 5/00; H05K 5/02

(52) U.S. Cl. CPC A61B 90/37; A61B 2090/372; H05K 5/0217; H05K 5/0017

(57) ABSTRACT

A supporting mechanism of an observation system for dental and medical treatment three-dimensionally movable supports a display unit for stereoscopically displaying an observation image of a surgical microscope regarding treatment of a patient on a dental chair and holds the display unit in an observation position of a dental surgeon keeping a constant posture. In a state where a vertically holding arm has been moved to a position where a vertical axial line thereof passes through an oral cavity of the patient, the supporting mechanism supports the display unit so as to be movable for positioning via a vertically fixed arm and a horizontally pivoting arm, in a position around the dental chair corresponding to a predetermined pivoting radius defined by a horizontal distance up to a finder of the display unit around the vertical axial line.

### BACKGROUND OF THE INVENTION

### Technical Field

The preset invention relates to an observation system for a dental and medical treatment for supporting a display unit for displaying an observation image of a surgical microscope or the like regarding a treatment of a patient on a chair so that the display unit can move three-dimensionally and holding the display unit in an observation position of a dental surgeon who keeps his/her fixed posture.

### Description of the Related Art

Conventionally, with the spread of a surgical microscope in the medical field, the surgical microscope is also introduced into dentistry, and a precise dental treatment using the surgical microscope is performed.

The conventional surgical microscope involves has a structural problem due to the fact that the surgical microscope is an optical microscope. Generally, the surgical microscope is composed of an objective lens and an eyepiece lens, and these lenses are optically coupled to each other and integrated. Therefore, if the objective lens is moved for changing an operating field, the eyepiece lens also moves together. Therefore, in order to look into the eyepiece lens, the dental surgeon is required to move together with the eyepiece lens or even to move a joint portion between the objective lens and the eyepiece lens.

Since the dental surgeon using the surgical microscope cannot keep the constant attitude in this manner, the dental treatment using the surgical microscope is very complicated, which results in an obstruction to the spread of the dental treatment using the surgical microscope. In view of these circumstances, a system where an image of a video camera attached to the surgical microscope is displayed on a two-dimensional monitor or a three-dimensional monitor and performs the dental treatment while viewing the image has been tried, but such a dental treatment is not widespread at all because it is difficult to observe the image with natural three stereoscopic vision.

On the other hand, a stereo viewer is adopted for an endoscopic surgery of the medical science. The stereo viewer is of a system where images of two video cameras attached to an endoscope are displayed on two monitors, respectively, and the dental surgeon views these images with his/her right and light respective eye. This provides very natural stereoscopic vision, as is the case with the eyepiece lens of the optical microscope, and is therefore widespread in the stereoscopic surgeries where fine treatment is required.

However, the conventional stereo viewer is considered to be unsuitable at all for the dental treatment where a treatment is performed within a narrow range on the face of a patient because the stereo viewer has a very large size, or has a small size but is remarkably poor in image quality.

By the way, in recent years, a need for a head-mounted stereo viewer is growing because of the spread of three-dimensional games or the like, and a product which is small in size but has a high quality is being developed, so that it is thought that the head-mounted stereo viewer is applied to the surgical microscope.

When the dental surgeon has tried to perform a dental treatment wearing the head-mounted stereo viewer electrically connected to the surgical microscope having right and left two video cameras attached, and succeeded in observation with natural stereoscopic vision, but, on the other hand, some problems occurred.

First of all, it was found that it is difficult for the dental surgeon to see a patient with the naked eye of the dental surgeon because the stereo viewer fixed to the head of the dental surgeon by the head mount is an obstruction. This inevitably involves a risk in view of the characteristic of the dentistry that the dental surgeon performs a treatment under the non-general anesthesia while being confirming the patient's reaction. Further, since the dental surgeon cannot easily take his/her eyes off from the stereo viewer, it is also difficult to take such an action as receiving/handing over an instrument, which results in the problem that the dental treatment using the head display becomes troublesome and time-consuming and the workload is also heavy.

### RELATED ART DOCUMENTS

### Patent Documents

Japanese Patent Application Laid-Open No. 2016-111533

Japanese Patent Application Laid-Open No. 2017-176318

Japanese Patent Application Laid-Open No. 2004-320722

Japanese Patent Application Laid-Open No. 2011-024820

### SUMMARY OF THE INVENTION

An object of the present invention is to provide an observation system for dental and medical treatment which can move a display unit for displaying an observation image for the treatment to a position optimum for the surgeon and hold the display unit.

(Observation System for Dental Treatment)

The present invention is directed to an observation system for dental and medical treatment, including:

a display unit for displaying a predetermined observation image regarding treatment of a patient on a dental chair; and

a supporting mechanism for supporting the display unit so as to be movable three-dimensionally and holding the display unit in an observation position of a dental surgeon keeping a constant posture, wherein

the supporting mechanism includes

a vertically holding arm supported so as to be movable to a position in which a vertical axial line passes through an oral cavity of the patient on the dental chair,

a horizontally pivoting arm having a rear end side supported on the vertically holding arm so as to be pivotable horizontally, and

a vertical arm provided downwardly at a distal end side of the horizontally pivoting arm, the display unit being attached to a distal end side of the vertical arm, and

the supporting mechanism has a structure where, in a state where the vertically holding arm has been moved to the position in which the vertical axial line of the vertically holding arm passes through an oral cavity of the patient, the display unit is supported so as to be movable for positioning via the vertical arm and the horizontally pivoting arm to a position around the dental chair corresponding to a predetermined pivoting radius defined by a horizontal distance up to a finder of the display unit around the vertical axial line.

(Vertically Pivoting Structure and Vertically Fixing Structure of Vertical Arm)

The vertical arm supporting the display unit at a distal end thereof is arranged at a distal end side of the horizontally pivoting arm so as to be pivotable about a vertical line or be fixed downwardly.

(Upward and Downward Turning Support of Display Unit)

The display unit is supported at a distal end of the vertical arm so as to be turnable upward and downward.

(Supporting Structure 1 on Base Side)

The supporting mechanism is provided with, between the base side and the vertically holding arm,

a second horizontally pivoting arm having a rear end side supported on the base side so as to be pivotable horizontally, and

a parallel link mechanism having a rear end side supported on a distal end side of the second horizontally pivoting arm so as to be pivotable horizontally for moving the vertically holding arm in three-dimensionally while keeping an attitude of the vertically holding arm provided on a distal end side of the parallel link mechanism.

(Supporting Structure 2 on Base Side)

The supporting mechanism is provided with, between the base side and the vertically holding arm, a parallel link mechanism having a rear end side supported on the base side so as to be pivotable horizontally for moving the vertically holding arm three-dimensionally while keeping a posture of the vertically holding arm provided on a distal end side of the parallel link mechanism.

(Supporting Structure 3 on Base Side)

The dental display supporting mechanism is configured such that a rear end side of the vertically holding arm is supported on the base side so as to be pivotable horizontally.

(Arm Telescopic Mechanism)

Either one or both of the horizontally pivoting arm and the vertically fixed arm are provided with a telescopic structure changing an arm length.

(Pivoting Radius of Display Unit)

The predetermined pivoting radius defined by the horizontal distance up to the finder of the display unit around the vertical axial line is set at a predetermined distance in a range from 10 cm to 30 cm.

(Advantageous Effect of the Observation System for Dental Treatment)

Since the present invention is directed to an observation system for dental and medical treatment, including: a display unit for displaying a predetermined observation image regarding treatment of a patient on a dental chair; and a supporting mechanism for supporting the display unit so as to be movable three-dimensionally and holding the display unit in an observation position of a dental surgeon keeping a constant posture, wherein the supporting mechanism includes a vertically holding arm supported so as to be movable to a position in which a vertical axial line passes through an oral cavity of the patient on the dental chair, a horizontally pivoting arm having a rear end side supported on the vertically holding arm so as to be pivotable horizontally, and a vertical arm provided downwardly at a distal end side of the horizontally pivoting arm, the display unit being attached to a distal end side of the vertical arm, and the supporting mechanism has a structure where, in a state where the vertically holding arm has been moved to the position in which the vertical axial line of the vertically holding arm passes through an oral cavity of the patient on a dental chair, the display unit is supported so as to be movable for positioning via the vertical arm and the horizontally pivoting arm to a position around the dental chair corresponding to a predetermined pivoting radius defined by a horizontal distance up to a finder of the display unit around the vertical axial line, when the dental surgeon treats a patient lying on the dental chair, viewing an image on the display unit (a stereoscopic image of the microscope), the dental surgeon consequently performs treatment from a surrounding position at a small distance from the face of the patient, for example, an optimum position between eight o'clock and four o'clock when the orientation of the head of the patient is twelve o'clock, but when the vertically holding arm of the supporting mechanism is positioned near just above the patient lying on the dental chair, the vertical axial line of the vertically holding arm is positioned so as to pass through the oral cavity of the patient, so that, in this state, by rotationally moving the display unit via the horizontally pivoting arm and the vertical arm so as to depict a circle centering around the oral cavity of the patient, the display unit can easily and securely be moved to and positioned in an optimum treatment position for the dental surgeon toward the face of the patient between eight o'clock and four o'clock.

(Advantageous Effect of Vertically Pivoting Structure and Vertically Fixing Structure of Vertical Arm)

Further, since the vertical arm supporting the display unit at a distal end thereof is arranged at the distal end side of the horizontally pivoting arm so as to be pivotable about a vertical line and be fixed downwardly, if the vertical arm is arranged at the distal end side of the horizontally pivoting arm so as to be pivotable about the vertical line, the display unit can be accommodated in a non-obstructive upper position by pivoting the vertical arm upwardly when the system is not in use. In addition, when the vertical arm is arranged fixedly at the distal end side of the horizontally pivoting arm, the display unit can easily and securely be pivoted horizontally without upward and downward movements when the display unit is pivotally moved around the dental chair.

(Advantageous Effect of Supporting Display Unit so as to be Turnable Upward and Downward)

Further, since the display unit is supported at the distal end of the vertical arm so as to be turnable upward and downward, the display unit is turned such that the finder side of the display unit is directed upward in accordance with the posture of the dental surgeon viewing the patient downward, so that the display unit can be positioned in an easily viewable position for the dental surgeon.

(Advantageous Effect of Supporting Structure 1 on Base Side)

Furthermore, since the supporting mechanism is provided with, between the base side and the vertically holding arm, a second horizontally pivoting arm having a rear end side supported on the base side so as to be pivotable horizontally, and a parallel link mechanism having a rear end side supported on a distal end side of the second horizontally pivoting arm so as to be pivotable horizontally for moving the vertically holding arm in three-dimensionally while keeping an attitude of the vertically holding arm provided on a distal end side of the parallel link mechanism, the rear end of the vertically holding arm is horizontally pivotally supported on a base fixed to, for example, a ceiling face via the parallel link mechanism and the second horizontally pivoting arm, so that the display unit can be moved three-dimensionally in a wide range, and the vertical axial line can easily be positioned in a position passing through the oral cavity of the patient by moving the vertically holding arm to just above the patient lying on the dental chair while maintaining an attitude of the vertically holding arm by the parallel link mechanism.

(Advantageous Effect of Supporting Mechanism 2 on Base Side)

Further, since the supporting mechanism is provided with, between the base side and the vertically holding arm, a parallel link mechanism having a rear end side supported on a distal end side of the horizontally pivoting arm so as to be pivotable horizontally for moving the vertically holding arm three-dimensionally while keeping a posture of the vertically holding arm provided on a distal end side of the parallel link mechanism, the vertically holding arm is moved to just above the patient lying on the dental chair while an attitude of the vertically holding arm is being maintained by the parallel link mechanism, so that the vertical axial line thereof can easily be positioned in a position passing through the oral cavity of the patient, and the supporting mechanism can be made compact and reduced in size and weight by an amount equivalent to the second horizontally pivoting arm removed, as compared with the supporting structure 1 on the base side described above.

(Advantageous Effect of Supporting Structure 3 on Base Side)

In addition, since the supporting mechanism is configured such that the rear end side of the vertically holding arm is supported on the base side so as to be pivotable horizontally such that the vertical axial line is located in a position passing through the oral cavity of the patient lying on the dental chair, an positioning operation of the vertically holding arm is made unnecessary by fixing the vertically holding arm such that the vertical axial line is located in a position passing through the oral cavity of the patient lying on the dental chair, and the supporting mechanism can be made compact and reduced in size and weight by an amount equivalent to the second horizontally pivoting arm and the parallel link mechanism removed, as compared with the supporting structure 1 on the base side described above, and by an amount equivalent to the parallel link mechanism removed, as compared with the supporting structure 2 on the base side described above.

(Telescopic Structure of Arm)

Further, since either one or both of the horizontally pivoting arm and the vertical arm are provided with a telescopic structure changing an arm length, the vertical axial line of the vertically holding arm can easily be moved to and accurately positioned in the position passing through the oral cavity of the patient lying on the dental chair by changing the arm length of the horizontally pivoting arm, and the height of the display unit can easily be adjusted to an appropriate height corresponding to treatment performed by the dental surgeon by changing the arm length of the vertically fixed arm.

(Advantageous Effect of Pivoting Radium of Display Unit)

Further, since the predetermined pivoting radius defined by the horizontal distance up to the finder of the display unit around the vertical axial line is set at a predetermined distance in a range from 10 cm to 30 cm, when the dental surgeon performs treatment while viewing an image on the display unit, the dental surgeon is prevented from excessively approaching the patient, and a sufficient space is secured above the patient, so that the dental surgeon can perform treatment while stereoscopically viewing an observation image of the patient's teeth on the display unit using a microscope without interference with the display unit or obstruction to the treatment performed the dental surgeon.

### BRIEF DESCRIPTION OF THE DRAWINGS

FIG. 1 is a descriptive diagram depicting an embodiment of an observation system for dental treatment using a surgical microscope having no eyepiece lens.

FIG. 2 is a descriptive diagram depicting a surgical microscope installed in a dental treatment room and a display unit supported by a supporting mechanism.

FIG. 3 is a descriptive diagram depicting a first embodiment of the supporting mechanism.

FIG. 4 is a descriptive diagram depicting a link configuration in the supporting mechanism shown in FIG. 3.

FIGS. 5A and 5B are descriptive diagrams depicting a display unit held at a distal end of a supporting arm.

FIG. 6 is a descriptive diagram depicting, in plan view, a position of the display unit held around a dental chair by the supporting mechanism shown in FIG. 3.

FIG. 7 is a descriptive diagram depicting an aspect of dental treatment according to an observation system for dental treatment provided with the supporting mechanism shown in FIG. 3

FIG. 8 is a descriptive diagram depicting the display unit moved to an accommodating position by the supporting mechanism shown in FIG. 3.

FIG. 9 is a descriptive diagram depicting holding of the display unit performed by the supporting mechanism shown in FIG. 3 when a patient's backrest is raised and a dental surgeon performs dental treatment in his/her standing position.

FIG. 10 is a descriptive diagram depicting the supporting mechanism shown in FIG. 3 where a horizontally pivoting arm on a base side is configured to have a telescopic structure.

FIG. 11 is a descriptive diagram depicting a second embodiment of a supporting mechanism where a rear end side of a parallel link arm is fixed to the base.

FIG. 12 is a descriptive diagram depicting a link configuration in the supporting mechanism shown in FIG. 11.

FIG. 13 is a descriptive diagram depicting a third embodiment of a supporting mechanism where a vertically holding arm is fixed to a base side and a vertical arm is configured to have a telescopic structure.

FIG. 14 is a descriptive diagram depicting a link configuration in the supporting mechanism shown in FIG. 13.

FIG. 15 is a descriptive diagram depicting a fourth embodiment of a supporting mechanism having a structure where a vertical arm supporting a display unit is fixed downwardly.

FIG. 16 is a descriptive diagram depicting a link configuration in the supporting mechanism shown in FIG. 15.

### DETAILED DESCRIPTION OF THE INVENTION

<Outline of Observation System for Dental Treatment>

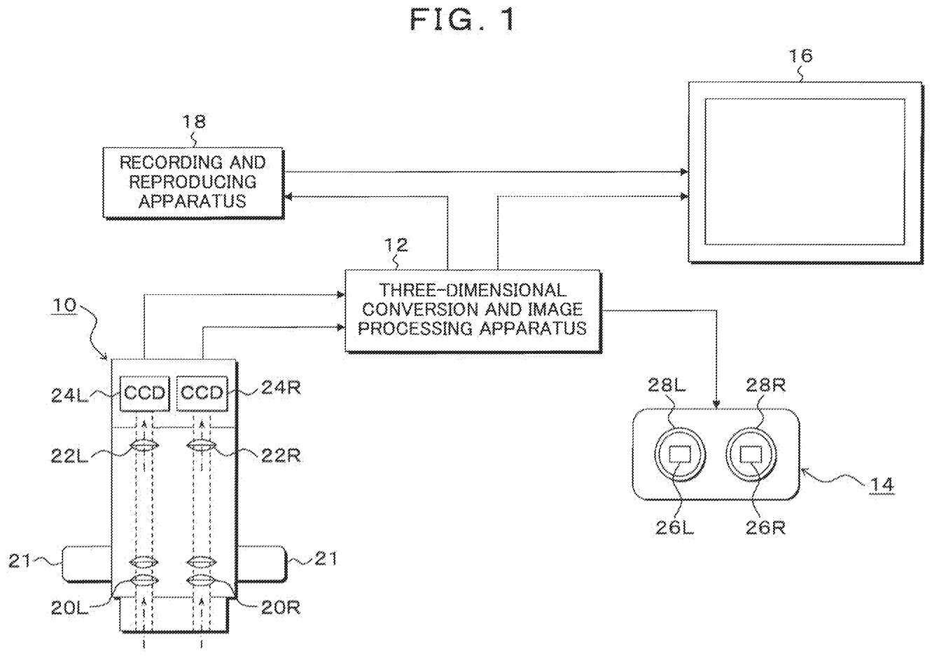

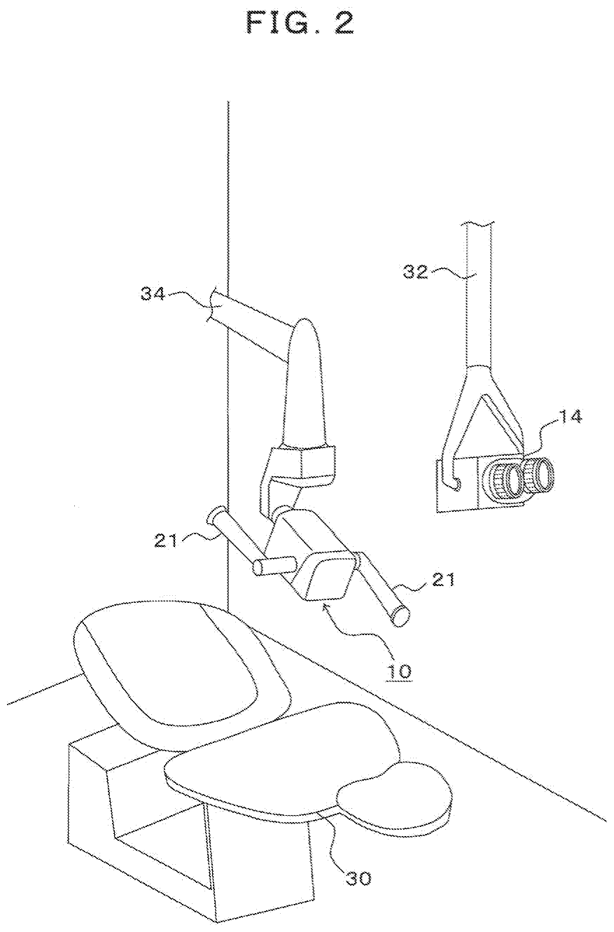

FIG. 1 is a descriptive diagram depicting an embodiment of an observation system for dental treatment using a surgical microscope not having an eyepiece lens, and FIG. 2 is a descriptive diagram depicting a surgical microscope installed in a dental treatment room and a display unit supported by a supporting arm.

As shown in FIG. 1, the observation system for dental treatment of the present embodiment is composed of a surgical microscope 10, a three-dimensional conversion and image processing apparatus 12, and a display unit (stereoscope) 14, and is provided with a monitor apparatus 16 and a recording and reproducing apparatus 18 if necessary.

As shown in FIG. 2, the surgical microscope 10 is supported on a dental chair 30 by a supporting mechanism 34 so as to be movable three-dimensionally, and a positioning operation thereof is performed by handling a handle 21.

Further, as shown in FIG. 1, the surgical microscope 10 of the present embodiment is an eyepiece lens-less type where focusing lenses 22L, 22R are arranged following two left and right objective lenses 20L and 20R, eyepiece lenses are not provided, and CCD video cameras 24L, 24R functioning as imaging devices are arranged at focusing positions of the focusing lenses 22L, 22R. The surgical microscope 10 shown in FIG. 2 is provided with binocular objective lenses, an illumination light and a filter on a lower face thereof opposed to the dental chair 30.

The CCD video cameras 24L, 24R each have a resolution of, for example, 1920×1080 pixels in vertical and horizontal directions, and a viewpoint video signal for the left eye is outputted from the CCD video camera 24L at 30 frames/second, and synchronously a viewpoint video signal for the right eye is outputted from the CCD video camera 24R. It is to be noted that the imaging device may be a CMOS video camera used instead of the CCD video camera.

The viewpoint video signals outputted from the CCD video cameras 24L, 24R are inputted to the 3D conversion and image processing apparatus 12 which in turn, as a 3D conversion function, converts the viewpoint video signals into a 3D-compatible video signal of a predetermined standard, for example, a side-by-side system comparable video signal to output the converted video signal to the display unit 14. The video signal from the three-dimensional conversion and image processing apparatus 12 is outputted according to an HDMI (registered trademark) protocol, so that the three-dimensional conversion and image processing apparatus 12 and the display unit 14 are connected to each other via an HDMI (registered trademark) cable.

The side-by-side system is a system for converting a video signal for the right eye and a video signal for the left eye into an interlaced video signal of 960×1080 pixels and transmitting the same, and use of a monitor device or a recording and reproducing apparatus comparable with the side-by-side system is made possible. It is to be noted that the three-dimensional conversion and image processing apparatus 12 may perform conversion into a 3D video signal of another system such as a frame packing system or a top-and-bottom system instead of the side-by-side system.

Further, the three-dimensional conversion and image processing apparatus 12, as the image processing function, synthesizes various digital images and outputs the synthetic image to the display unit 14 and also performs predetermined image processing, such as enhancement of a specific color tone or exposure correction, to the viewpoint video signals from the pair of CCD video cameras 24L, 24R obtained by irradiating the operating field with light of a specific wavelength with a filter or the like interposed between the operating field and the light source of the surgical microscope 10. By this image processing, the dental surgeon can perform treatment stereoscopically viewing, on the display unit 14, a microscopic observation image obtained from the viewpoint video signals after the image processing where a lesion or an anatomical from which is such as to be difficult to discriminate with the naked eye has been visualized.

The display unit 14 is of a binoculars type in this embodiment, and has a pair of image display devices 26L, 26R arranged in positions corresponding to the left eye and the right eye of the dental surgeon within lens barrels 28L, 28R, and displays the left and right viewpoint video signal images converted by the three-dimensional conversion and image processing apparatus 12 on the pair of image display devices 26L, 26R, thereby making the dental surgeon view an observation image of the surgical microscope 10 stereoscopically.

As the image display devices 26L, 26R, for example, an organic EL panel having a screen of 5.7 inches and having vertical and horizontal 1980×1080 pixels is divided into left and right portions and used, or two left and right small-sized image display devices are used. Further, an LED panel or a liquid crystal panel having a resolution equivalent to the resolution of the organic EL panel may be used instead of the organic EL panel.

As shown in FIG. 2, the display unit 14 of this embodiment is supported at a distal end of the supporting mechanism 32, and it is held in an observation position of the dental surgeon sitting on a chair and keeping his/her constant posture for treating the patient lying on the dental chair 30. Thereby, the dental surgeon can carry on dental treatment confirming the microscopic image on the display unit 14 with stereoscopic vision while maintaining his/her constant posture (fixed) without moving himself/herself even if the dental surgeon moves the surgical microscope 10 relative to the movement of the operating field or the patient.

Furthermore, though the dental surgeon looks away from the display unit 14 in order to confirm reaction of the patient under non-general anesthesia, since the display unit 14 is held by the supporting frame 32, the movement of the dental surgeon is not constrained so that the dental surgeon can look away from the display unit 14 to confirm the patient easily. Further, the dental surgeon can also look away from the display unit 14 to facilitate such an action as receiving/handing over an instrument from/to his/her assistant. As a result, the dental surgeon can carry on precise dental treatment efficiently confirming the microscopic image on the display unit 14 with stereoscopic vision.

The monitor apparatus 16 is arranged on a wall of the treatment room, if necessary, and the left and right binocular viewpoint video signals outputted from the three-dimensional conversion and image processing apparatus 12 are inputted to the monitor apparatus 16, and the monitor apparatus 16 displays the microscopic observation image as a three-dimensional image or two-dimensional image according to the side-by-side system, so that a person concerned such as the dental surgeon or the assistant can observe the image.

The recording and reproducing apparatus 18 is arranged, if necessary, and are inputted to the recording and reproducing apparatus 18 the left and right binocular viewpoint video signals outputted from the three-dimensional conversion and image processing apparatus 12, and the recording and reproducing apparatus 18 records the microscopic observation image as a three-dimensional image or two-dimensional image according to the side-by-side system, and also reproduces the same, if necessary, so that it is made possible to display the microscopic observation image recorded on the monitor apparatus 16 as a three-dimensional image or two-dimensional image.

It is to be noted that, in the present embodiment, the viewpoint video signals from the CCD video cameras 24L, 24R provided in the surgical microscope 10 are converted into, for example, the viewpoint video signal of the side-by-side system by the three-dimensional conversion and image processing apparatus 12, but the three-dimensional conversion and image processing apparatus 12 may not be provided and instead the viewpoint video signals from the CCD video cameras 24L, 24R may be directly transmitted to the image display devices 26L, 26R of the display unit 14 to make the image display devices 26L, 26R display viewpoint images, thereby making the dental surgeon stereoscopically view the microscopic observation image. However, without the three-dimensional conversion/image processing apparatus 12 provided, it is impossible to deal with the monitor apparatus 16 or the recording and reproducing apparatus 18, it is desirable to provide the three-dimensional conversion and image processing apparatus 12.

It is to be noted that the optical microscope has the eyepiece lenses through which an observation is made, whereas a digital microscope of the observation system for dental treatment of the present embodiment is provided with image display devices serving as the eyes through which an observation is made on the liquid crystal monitor, and such a digital microscope includes an optical fiberscope provided with lenses at distal ends of a pair of optical fibers and allowing an observation on the liquid crystal monitor of an image taken by imaging devices provided on rear end sides of the optical fibers.

<First Embodiment of Supporting Mechanism>

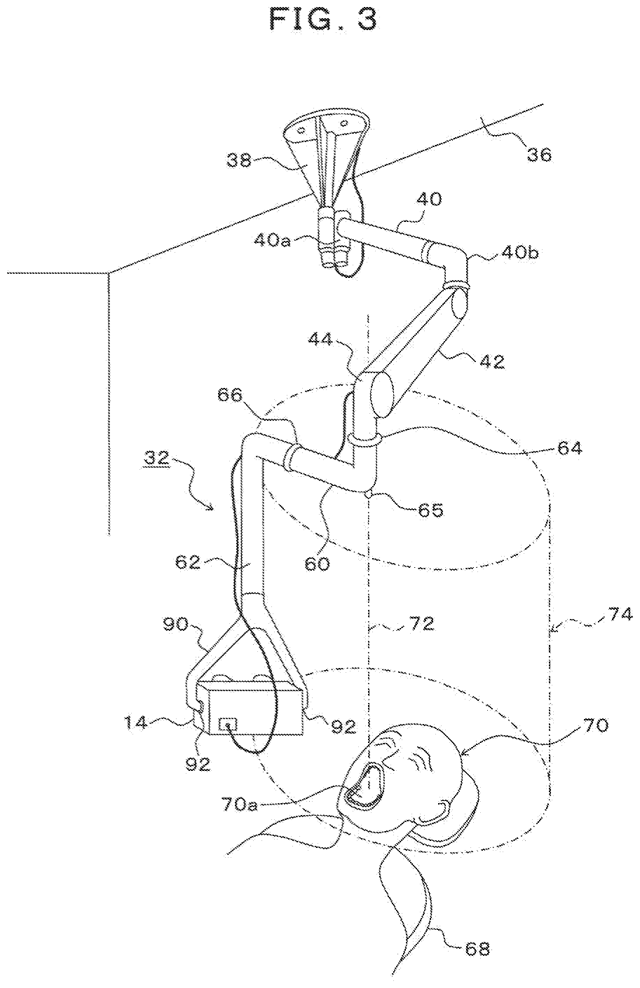

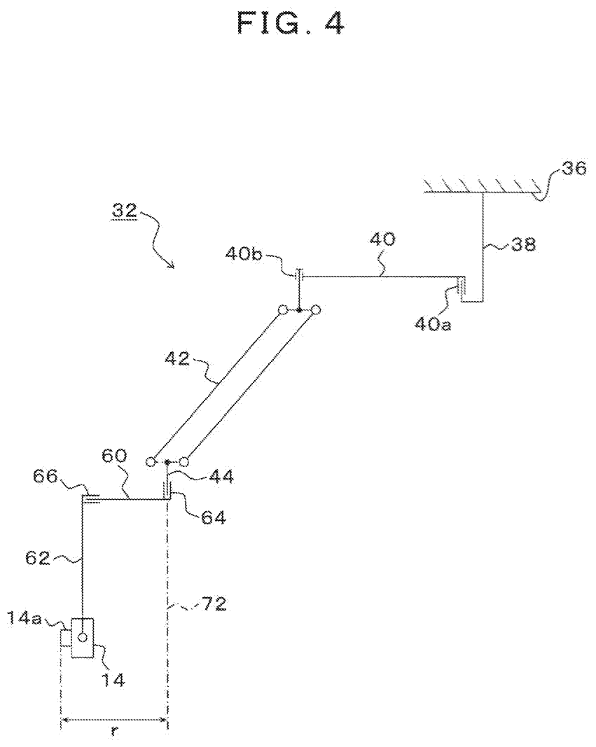

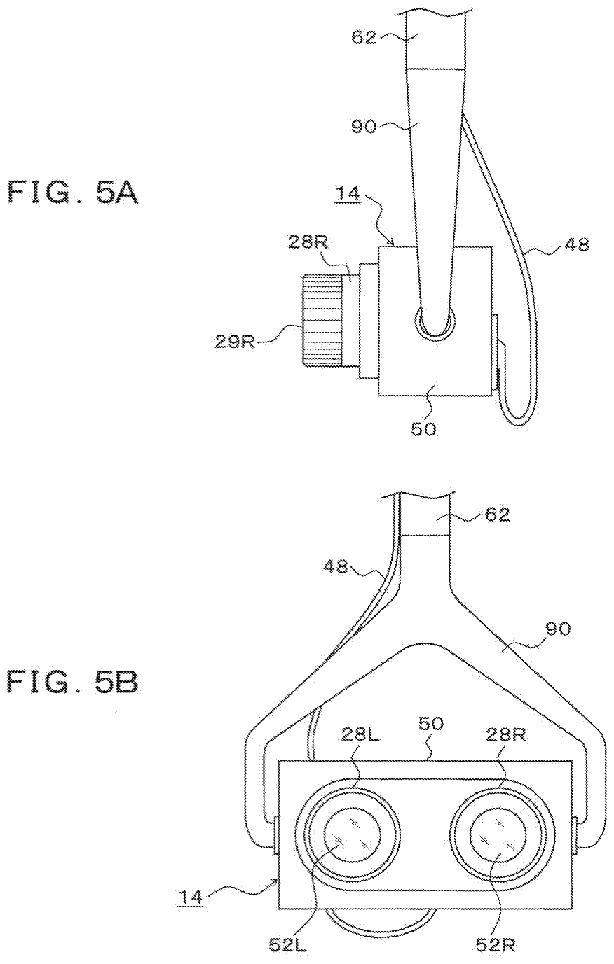

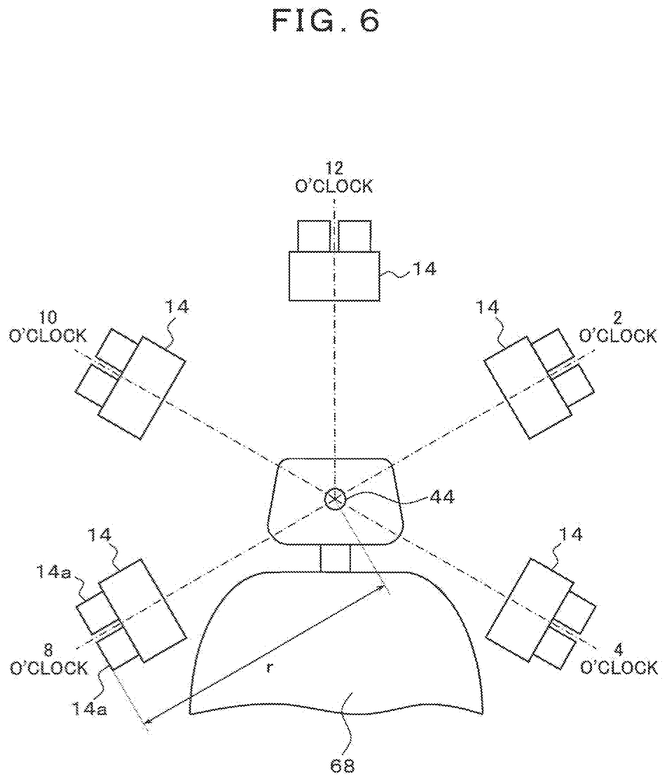

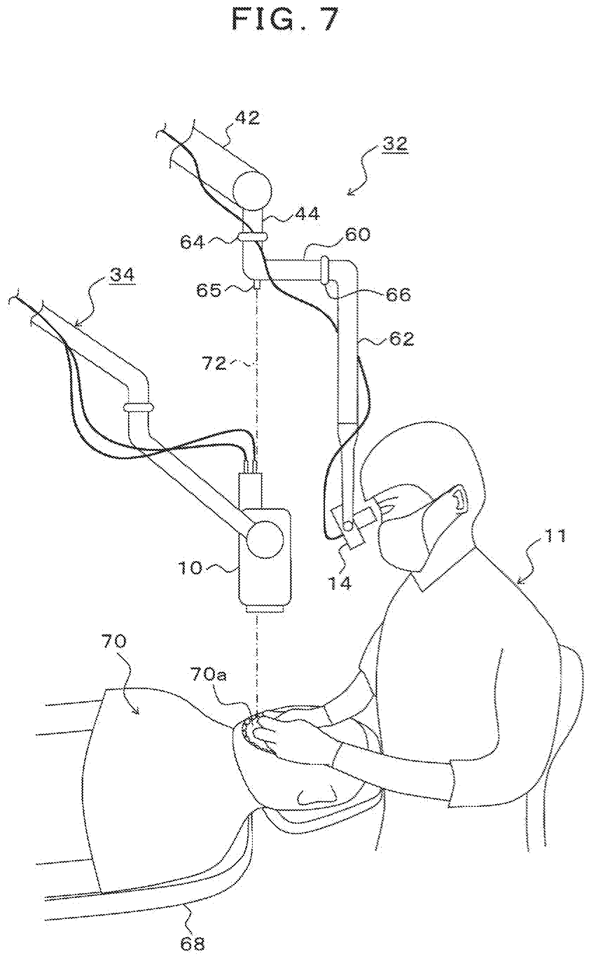

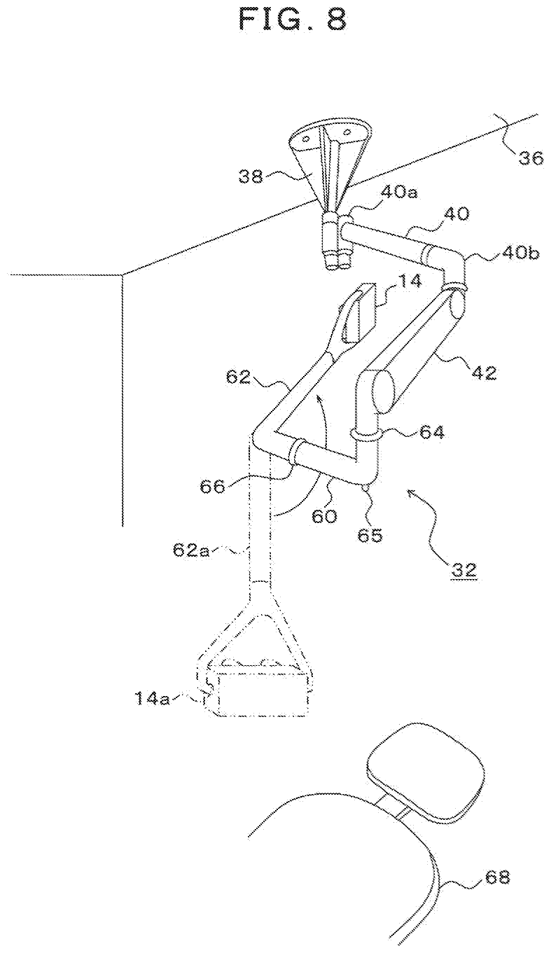

FIG. 3 is a descriptive diagram depicting a second embodiment of the supporting mechanism, FIG. 4 is a descriptive diagram depicting a link configuration in the supporting mechanism depicted in FIG. 3, and FIGS. 5A and 5B are descriptive diagrams depicting a display unit held at a distal end of a vertically fixed arm, and FIG. 5A is a side view of the display unit and FIG. 5B is a front view thereof. FIG. 6 is a descriptive diagram depicting, in plan view, a position of the display unit held around the dental chair by the supporting mechanism depicted in FIG. 3, FIG. 7 is a descriptive diagram depicting an aspect of dental treatment performed by the observation system for dental treatment provided with the supporting mechanism depicted in FIG. 3, and FIG. 8 is a descriptive diagram depicting the supporting mechanism depicted in FIG. 3 holding the display unit when a dental surgeon performs dental treatment in his/her standing position with a backrest for the patient raised.

(Configuration of Supporting Mechanism)

As shown in FIG. 3, a supporting mechanism 32 of the present embodiment is composed of a base 38, a horizontally pivoting arm 40 functioning as a second horizontally pivoting arm, a parallel link arm 42, and a vertically holding arm 44, which constitute the same link configuration as the first embodiment depicted in FIG. 3, and are followed by a horizontally pivoting joint 64, a horizontally pivoting arm 60, a vertically pivoting joint 66 and a vertical arm 62.

That is, the supporting mechanism 32 of the present embodiment configures a parallel link mechanism in which a rear end side of the horizontally pivoting arm 40 is supported on the base 38 fixed on, for example, a ceiling 36 so as to be pivotable horizontally on the horizontally pivoting joint 40a, a rear end side of the parallel link arm 42 is supported on a distal end side of the horizontally pivoting arm 40 so as to be pivotable horizontally on the horizontally pivoting joint 40b, and the parallel link arm 42 moves the vertically holding arm 44 at a distal end side of the parallel link arm 42 in three-dimensionally maintaining the attitude of the vertically holding arm 44.

The horizontally pivoting arm 60 is supported on the vertically holding arm 44 fixed to the distal end side of the parallel link arm 42 so as to be pivotable horizontally by the horizontally pivoting joint 64, subsequently the vertical arm 62 is supported on a distal end side of the horizontally pivoting arm 60 so as to be pivotable vertically on the vertically pivoting joint 66, on a distal end side of the vertical arm 62, both sides of the display unit 14 are supported on shaft portion 92 of distal ends of forked supporting arms 90 so that the display unit 14 is attached so as to be capable of upward and downward angular adjustment.

It is to be noted that a center of a top of the display unit 14 may be fixed to the distal end (lower end) of the vertically fixing arm 62 instead of the forked supporting arms 90. Alternatively, the distal end of the vertical arm 62 may be bent laterally and fixed to either one of left and right side faces of the display unit 14, or the distal end of the vertical arm 62 may be bent upward fixed to a bottom face of the display unit 14.

A light beam emitting portion 65 using light emission of an LED or the like to produce a bright spot for locating by irradiating the patient with such a weak light beam in a direction of a vertical axial line 72 as to give no harmful effect on the patient is provided at a lower end of the horizontally pivoting arm 60 through which the vertical axial line 72 of the vertically holding arm 44 passes, namely, an arm lower end site through which the vertical axial line 72 of the vertically holding arm 44 passes.

Since the light beam emitting portion 65 is used, when the vertically holding arm 64 is moved to just above the patient 70 lying on the dental chair 68, the vertical axial line 72 of the vertically holding arm 44 can be easily and accurately set in a position passing through an oral cavity 70a of the patient 70 by performing adjustment such that the bright spot of the light beam from the light beam emitting portion 65 hits on the oral cavity 70a of the patient 70. The light beam emitting portion 65 can be turned on and off by operating a switch (not depicted), and is used by turning on when the vertically holding arm 44 is located and put off by turning off the switch after the locating is completed.

It is to be noted that, even without using the light beam emitting portion 65, when the dental surgeon moves the vertically holding arm 44 to just above the patient 70 lying on the dental chair 68, the vertical axial line 72 of the vertically holding arm 44 can be located in the position passing through the oral cavity 70a of the patient 70, so that the light beam emitting portion 65 may not necessarily be provided.

(Display Unit)

As shown in FIGS. 5A and 5B, the display unit 14 of the present embodiment is provided with a pair of left and right lens barrels 28L, 28R on a front face of a unit main body 50 having a thin box shape, provided with diopter adjustment dials 29L, 29R capable of adjusting an interpupillary distance and a diopter on the lens barrels 28L, 28R, and further provided with hooded cover glasses 52L, 52R at distal ends of the lens barrels 28L, 28R, and the display unit 14 makes it possible view the microscopic observation image stereoscopically through the image display devices using an organic EL panel or the like arranged within the unit main body 50.

The unit main body 50 of the display unit 14 is attached vertically below the vertical arm 62, and the dental surgeon brings his/her eyes close to the cover glasses 52L, 52R of the lens barrels 28L, 28R projecting forward from the unit main body 50, thereby being capable to perform treatment viewing the microscopic observation image stereoscopically on the display unit 14 arranged spatially without being obstructed by the surgical microscope.

Further, since the supporting mechanism 32 is extending down from an upper space above the dental surgeon, when the dental surgeon looks away from the display unit 14 and confirms the patient or when the dental surgeon receives or hands over an instrument, the dental surgeon can carryon precise dental treatment efficiently viewing the microscopic observation image stereoscopically without being obstructed by the supporting mechanism 32.

It is to be noted that the display unit 14 of the present embodiment is merely an example, and it is not limited to the binoculars-type one, and includes a display unit having an appropriate structure as long as the display unit is configured such that a pair of image display devices arranged in positions corresponding to both eyes of the dental surgeon display left and right viewpoint video signal images to make the dental surgeon viewing the observation image of the surgical microscope stereoscopically.

(Action of Supporting Mechanism)

As shown in FIG. 3, when the patient 70 lying on the dental chair 68 is treated, the parallel link arm 42 is moved and positioned such that the vertically holding arm 44 in the supporting mechanism is located just above the oral cavity 70a of the patient 70. In this state, the vertical axial line 72 of the vertically holding arm 44 can be set in the position passing through the oral cavity 70a of the patient 70 lying on the dental chair 68 by performing adjustment such that the bright spot produced by the light beam from the light beam emitting portion 65 hits on the oral cavity 70a of the patient 70.

In this state, the display unit 14 is supported so as to be pivotable about a horizontal line to the vertically holding arm 44 via the vertical arm 62 and the horizontal pivoting arm 60, and as shown in FIG. 6, the display unit 14 can be pivotally moved around the dental chair 68 so as to make a circle having a radius r centering on the vertical axial line 72 by determining the length of the horizontally pivoting arm 60 such that a distance from the vertical axial line 72 to a finder 14a of the display unit 14 is a predetermined pivoting radius r.

In the present embodiment, when the dental surgeon sits on a chair, and treats the patient 70 lying on the dental chair 68 while viewing a stereoscopic image obtained by the surgical microscope on the display unit 14, the dental surgeon performs the treatment moving to an optimum position, if necessary, between surrounding positions at a small distance from the face of the patient 70, for example, between eight o'clock and four o'clock when the orientation of the head of the patient is twelve o'clock, as shown in FIG. 6. It is to be noted that, though the assistant usually takes his/her position of four o'clock, the dental surgeon may take his/her position at four o'clock.

When the vertically holding arm 44 on the distal end side of the parallel link arm 42 is positioned close to just above such the patient 70 lying on the dental chair 68 in response to movement of the dental surgeon to the patient 70 lying on the dental chair 68, as shown in FIG. 3, the vertical axial line 72 of the vertically holding arm 44 can be set approximately at the position passing through the oral cavity 70a of the patient 70. In this state, the display unit 14 can easily and securely be positioned in an optimal treatment position toward the face of the patient between eight o'clock and four o'clock shown in FIG. 5 by pivotally moving the display unit 14 so as to encircle the patient 70 via the vertical arm 62 and the horizontal pivoting arm 60.

For example, as shown in FIG. 7, when the dental surgeon 11 sitting the chair moves to a back position corresponding to twelve o'clock shown in FIG. 6 and performs treatment, the dental surgeon positions the vertically holding arm 44 of the supported mechanism 32 just above the patient 70 to direct the axial line 72 of the vertically holding arm to the oral cavity 70a of the patient 70, and in this state, he/she moves the display unit 14 to the position of twelve o'clock and holds the same in the position and displays an image of teeth of the patient 70 taken by the imaging device provided in the surgical microscope 10 on the display unit 14 so that the dental surgeon can carry on the dental treatment stereoscopically viewing the teeth image displayed on the display unit 14.

Here, the dental surgeon 11 is performing the treatment in a surrounding position at a distance of approximately 10 to 30 cm from the face of the patient 70 lying on the dental chair 68. Therefore, in the supporting mechanism 32 of the present embodiment, the length of the horizontally pivoting arm 60 is set such that the radius r which is a distance from the central vertical axial line 72 to the finder 14a of the display unit 14 is a predetermined value within a range of about 10 cm to 30 cm, for example, the radius r is about 20 cm.

In this manner, a circle having the radius r on which the display unit 14 moves around the vertical axial line 72 is determined by the length of the horizontal pivoting arm 60, and, as depicted in FIG. 3, a virtual cylindrical empty space 74 having a top plane defined by horizontal rotation of the horizontal pivoting arm 60 and a bottom plane defined by horizontal rotation of the display unit 14 is formed above the patient 70.

This cylindrical empty space 74 is a space for the dental surgeon to treat the patient 70 without interference with movement of the display unit 14 from eight o'clock to four o'clock from another member or equipment and a space in which the surgical microscope 10 is arranged and which is used for observing the oral cavity 70a of the patient 70 can also be secured, as shown in FIG. 6.

Further, in the treatment using the display unit 14, the dental surgeon frequently repeats treatment using the display unit 14 and treatment performed viewing the oral cavity 70a of the patient 70 with the naked eye without using the display unit 14, and when the dental surgeon does not use the display unit 14, the dental surgeon can perform treatment with the naked eye by pushing the display unit 14 rightward or leftward to move the display unit 14 quickly rightward or leftward integrally with the vertically fixed arm 62 and the horizontally pivoting arm 60 about the vertical holding arm 44, and the dental surgeon can also return the display unit 14 quickly to the original position.

At this time, as shown in FIG. 7, the surgical microscope 10 is held by another supporting mechanism 34, and the dental surgeon can leave the surgical microscope 10 as it is and move only the display unit 14 easily to a position for treatment with the naked eye moved leftward or rightward from an observation position in front of the dental surgeon 11.

(Accommodation of Supporting Mechanism)

Furthermore, in a case where the microscope system of the present embodiment is not introduced, the ceiling of a dental treatment room is not very high for various reasons such as a need for plumbing beneath the floor of the dental treatment room, and when a patient or a staff or someone else enters or leaves, the supporting mechanism 32 of the display unit 14 is often obstructive. On the other hand, as shown in FIG. 8, the supporting mechanism 32 of the present embodiment is folded from a position indicated by vertical arm 62a in use to an upward rotated position of the vertical arm 62 at approximately the same level as the parallel link arm 42 so that the display unit 14 can be accommodated in a non-obstructive position.

(Dental Treatment of Patient in Standing Position)

The positions of a patient during dental treatment include a horizontal position in which the patient 70 is lying on the dental chair 38, as shown in FIG. 3, and a standing position in which the patient 70 is sitting on the dental chair with the backrest raised. When the patient is in the horizontal position, the dental surgeon is sitting on a chair, and he/she can perform dental treatment stereoscopically viewing the teeth of the patient observed by the surgical microscope 10 on the display unit 14.

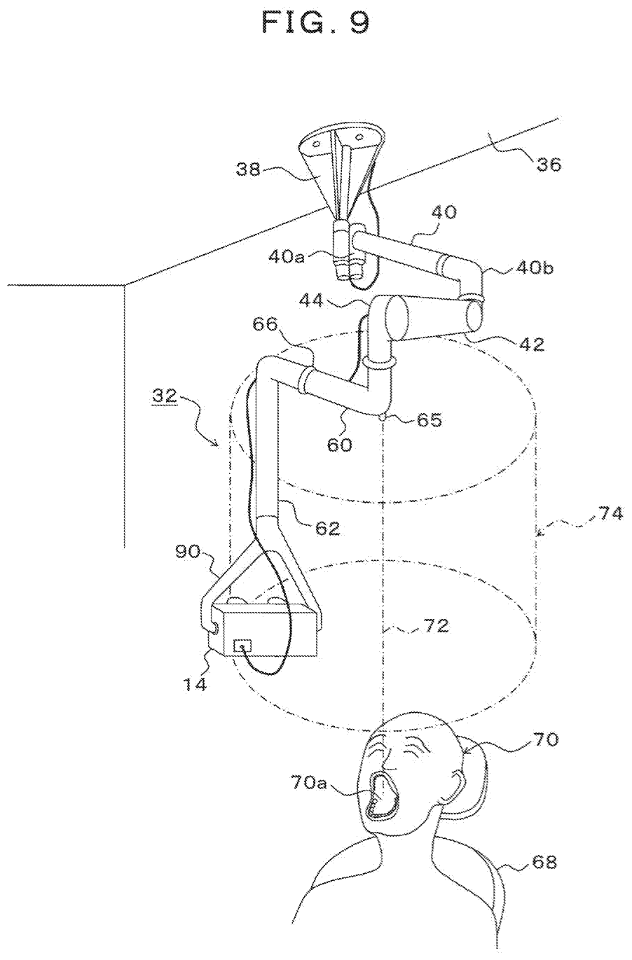

However, as shown in FIG. 9, for an aged person who is likely to aspirate or ingest foreign matter accidentally or a patient who is coughing or feels sick, the dental surge on may perform dental treatment to the patient 70 in a standing position with the backrest of the dental chair 68 raised. In this case, in the conventional dental treatment directly through the surgical microscope, since the eyepiece lens is located in a low position, the dental surgeon is forced to perform treatment viewing the surgical microscope in his/her half-sitting posture, and he/she cannot perform dental treatment using the surgical microscope to the patient in the standing position.

On the other hand, in the supporting mechanism 32 of the present embodiment, as shown in FIG. 8, also for the patient 70 in the standing position, in the same manner as in the horizontal position, the vertically holding arm 44 of the supporting mechanism 32 is positioned just above the patient 70 to direct the vertical axial line 72 of the vertically holding arm 44 to the oral cavity 70a of the patient 70, and thereby the display unit can be pivoted on the vertical axial line 72 around the patient 70 via the vertical arm 62 and the horizontally pivoting arm 60 and held in any position required for treatment, and the height of the display unit 14 can easily be held in a position at the eye level of the dental surgeon standing around the patient 70, so that the dental surgeon can easily and appropriately proceed to dental treatment to the patient in the standing position, without taking such an unnatural posture as a half-sitting posture, stereoscopically viewing the observation image of the surgical microscope on the display unit 14.

(Telescopic Structure of Horizontally Pivoting Arm on Base Side)

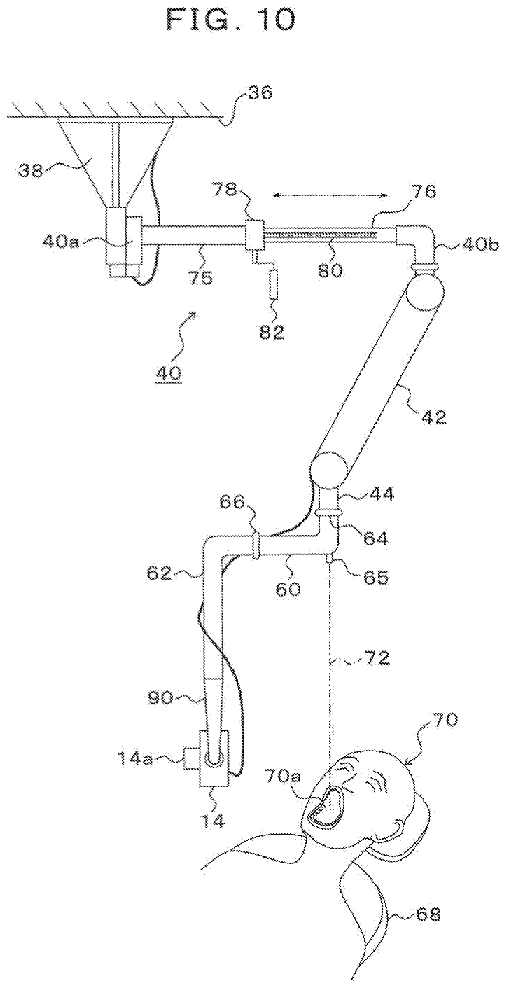

FIG. 10 is a descriptive diagram depicting the supporting mechanism depicted in FIG. 3 where the horizontally pivoting arm on the base side has a telescopic structure.

As shown in FIG. 10, the supporting mechanism 32 of the present embodiment is configured such that the horizontally pivoting arm 40 coupled horizontally pivotally to the base 38 fixed to a ceiling face 36 has a telescopic structure. The telescopic structure of the horizontally pivoting arm 40 is configured such that a cylindrical fixed arm 74 is coupled to the horizontal pivoting joint 40a on the base 38 side and a telescopic arm 76 is fitted into the fixed arm 75 so as to be slidable in an axial direction.

A telescopic gear mechanism 78 is provided at a distal end side of the fixed arm 75, a pinion gear (not shown) contained therein can be rotated by an external handle 82, and the pinion gear meshes with a rack gear 80 formed axially on an outer periphery of the telescopic arm 76.

When the handle 82 is turned, for example, clockwise, the rack gear 80 is drawn into the fixed arm 75 by clockwise rotation of the pinion gear so that the horizontally pivoting arm 40 can be shortened, and when the handle 82 is reversely turned counterclockwise, the rack gear 80 is pushed out of the fixed arm 75 by counterclockwise rotation of the pinion gear so that the horizontally pivoting arm 40 can be lengthened. Since the other structures and functions are the same as those of the second embodiment depicted in FIG. 7, same reference numerals are attached thereto and explanations thereof are omitted.

Since the length of the horizontally pivoting arm 40 can be changed in this manner, regardless of variations among dental treatment rooms in terms of a position in which the base 38 can be fixed to the ceiling face 36 the vertically holding arm at the distal end of the parallel link arm 42 can easily be positioned reliably above the oral cavity 70a of the patient 70 in the horizontal position on the dental chair 68, and is held so as to direct the vertical axial line 72 of the vertically holding arm 44 to the oral cavity 70a of the patient 70, so that the display unit 14 can easily be moved on the vertical axial line 72 to held in an any position from eight o'clock to four o'clock around the patient 70 via the horizontally pivoting arm 60 and the vertical arm 62.

Further, since the vertically holding arm 44 is positioned just above the patient 70 by changing the length of the horizontally pivoting arm 40 such that the vertical axial line 72 is directed to the oral cavity 70a of the patient 70, when a transition is made from the treatment under the microscope using the display unit 14 to the treatment with the naked eye, the dental surgeon can quickly move the display unit 14 by the counterclockwise or clockwise horizontal rotation of the display unit 14 on the vertically holding arm 44 and can also quickly return the display unit 14 to the original position to go back to the treatment under the microscope.

It is to be noted that, though the telescopic structure of the horizontally pivoting arm 40 adopts manual operation with the handle 82, an actuator such as a motor may be provided so that the length of the horizontally pivoting arm 40 can be changed by remote control.

<Second Embodiment of Supporting Mechanism>

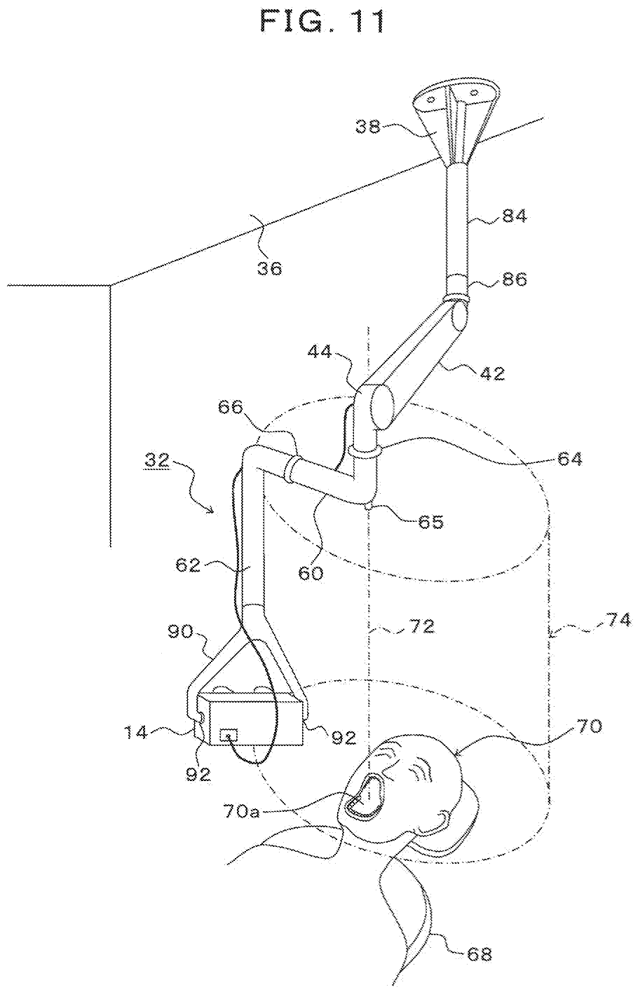

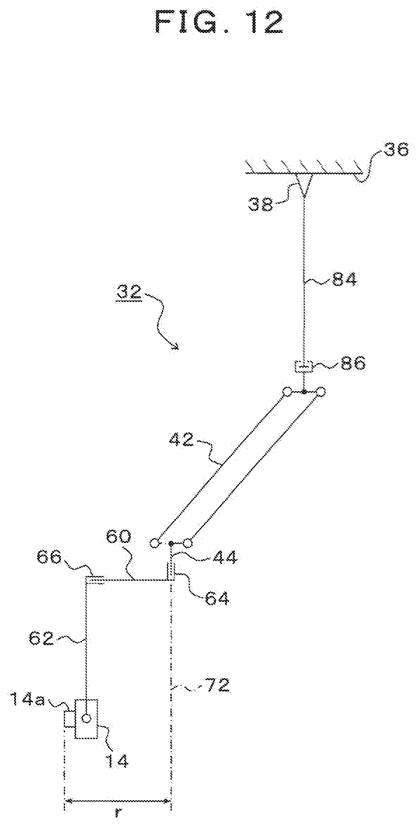

FIG. 11 is a descriptive diagram depicting a second embodiment of a supporting mechanism where a rear end side of a parallel link arm is fixed to a base, and FIG. 12 is a descriptive diagram depicting a link configuration in the supporting mechanism depicted in FIG. 11.

As depicted in FIG. 11 and FIG. 12, the supporting mechanism 32 of the present embodiment is characterized by using the mechanism following the parallel link arm 42 in the supporting mechanism 32 of the first embodiment depicted in FIG. 3.

That is, the supporting mechanism 32 of the present embodiment is configured such that a rear end of the parallel link arm 42 is coupled via a horizontally pivoting joint 86 to a lower end of a fixed arm 84 fixed downwardly to the base 38 fixed to the ceiling face 36, and what follows the parallel link arm 42 are the same as those of the first embodiment depicted in FIG. 3, so that the display unit 14 is attached via the shaft portions 92 to the forked supporting arm 90 fixed at the distal end of the vertical arm 62 to the parallel link arm 42 via the vertically holding arm 44, the horizontally pivoting arm 60 and the vertical arm 62.

Since the supporting mechanism 32 of the present embodiment is not provided with the horizontally pivoting arm 40 depicted in FIG. 3 on the base 38 side, the supporting mechanism 32 is compact accordingly as a whole, and has a structure suitable for a dental treatment room where it is difficult to secure a pivoting range of the horizontally pivoting arm 40.

Further, though the supporting mechanism 32 is compact, by using the parallel link arm 42 to position the vertically holding arm 44 just above the patient 70 in the horizontal position and hold the vertical axial line 72 of the vertically holding arm 44 directed to the oral cavity 70a of the patient 70, so that the display unit 14 can easily be moved pivotally on the vertical axial line 72 via the horizontally pivoting arm 60 and the vertical arm 62 and held in an any position from eight o'clock to four o'clock around the patient 70.

Further, the empty space 74 enabling the dental surgeon to treat the patient 70 without interference with movement of the display unit 14 from eight o'clock to four o'clock from another member or equipment and, as depicted in FIG. 10, used to observe the oral cavity 70a of the patient 70 through the surgical microscope 10 positioned in the empty space can be secured above the patient 70.

<Third Embodiment of Supporting Mechanism>

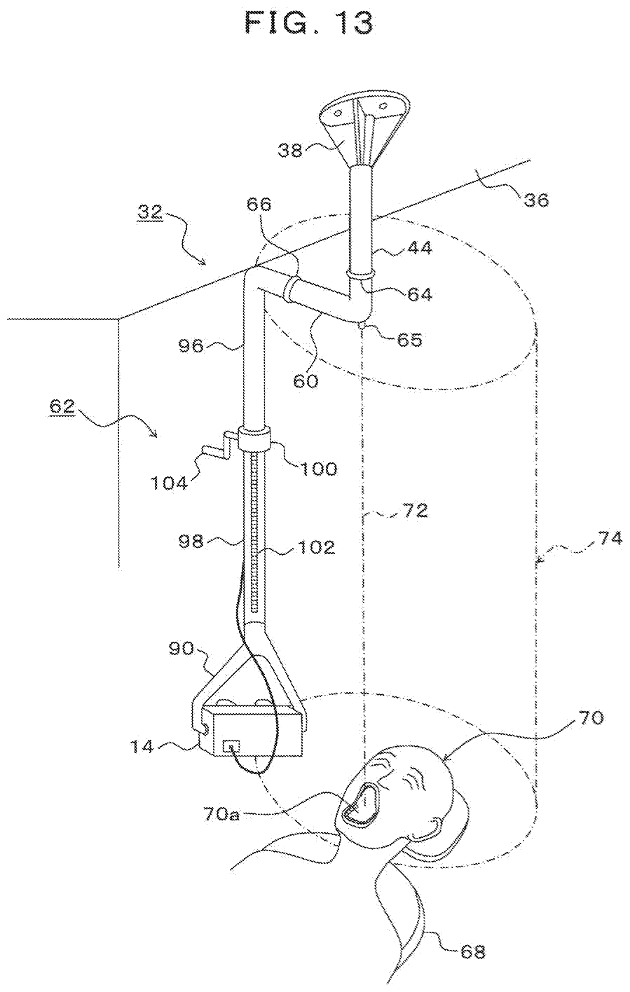

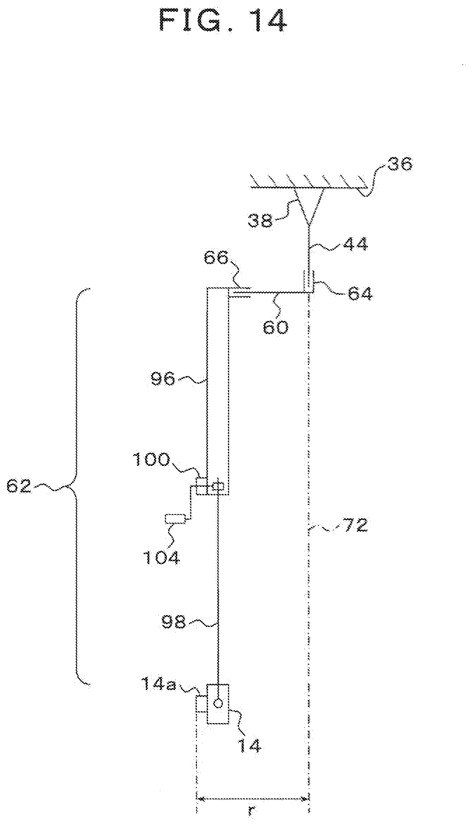

FIG. 13 is a descriptive diagram depicting a third embodiment of the supporting mechanism where the vertically holding arm is fixed to the base side and the vertical arm has a telescopic structure, and FIG. 14 is a descriptive diagram depicting a link configuration in the supporting mechanism depicted in FIG. 13.

As depicted in FIG. 13 and FIG. 14, the supporting mechanism 32 of the present embodiment is characterized by using the mechanism following the vertically holding arm 44 in the supporting mechanism 32 of the first embodiment depicted in FIG. 3.

That is, the supporting mechanism 32 of the present embodiment is configured such that the vertically holding arm 44 is fixed downwardly to the base 38 fixed to the ceiling face 36, and what follow the vertically holding arm 44 are basically the same as those of the second embodiment depicted in FIG. 3, so that the display unit 14 is attached via the shaft portions 92 to the forked supporting arm 90 fixed at the distal end of the vertical arm to the vertically holding arm 44 via the horizontally pivoting arm 60 and the vertical arm 62.

Further, the vertical arm 62 of the present embodiment has a telescopic structure. The telescopic structure of the vertical arm 62 is configured such that a cylindrical fixed arm 96 is coupled to a distal end of the horizontally pivoting arm 60 via the vertically pivoting joint 66 and a telescopic arm 98 is fitted in the fixed arm 96 so as to be slidable in an axial direction (vertical direction).

A telescopic gear mechanism 100 is provided at a distal end side of the fixed arm 96, and a pinion gear (not shown) contained therein can be rotated by an external handle 104, and the pinion gear meshes with a rack gear 102 axially formed on an outer periphery of the telescopic arm 98.

When the handle 104 is turned, for example, clockwise, the rack gear 102 is pulled up into the fixed arm 96 by clockwise rotation of the pinion gear so that the vertical arm 62 can be shortened, and when the handle 104 is reversely turned counterclockwise, the rack gear 102 is pushed down from the fixed arm 96 by counterclockwise rotation of the pinion gear so that the vertical arm 62 can be lengthened.

Since the length of the vertical arm 62 can be changed in this manner, regardless of variations among dental treatment rooms in terms of a position in which the base 38 can be fixed to the ceiling face 36, the vertically holding arm 44 at the distal end of the parallel link arm 42 can easily be positioned reliably above the oral cavity 70a of the patient 70 in the horizontal position on the dental chair 68, and is held so as to direct the vertical axial line 72 of the vertically holding arm 44 to the oral cavity 70a of the patient 70, so that he display unit 14 can easily be moved pivotally on the vertical axial line 72 via the horizontally pivoting arm 60 and the vertical arm 62 and held in an any position from eight o'clock to four o'clock around the patient.

Since the supporting mechanism 32 of the present embodiment does not include the horizontally pivoting arm 40 and the parallel link arm 42 depicted in FIG. 3 on the base 38 side, the supporting mechanism is considerably compact as a whole, and since it is necessary to secure the pivoting range of the horizontally pivoting arm 60, an installation space for the supporting mechanism 32 can be reduced significantly.

Further, in a case where the supporting mechanism 32 of the present embodiment is installed in the dental treatment room, the base 38 is fixed to the ceiling face 36 such that the vertical axial line 72 from the vertically holding arm 44 is directed to the oral cavity 70a of the patient 70 in the horizontal position.

Therefore, since the vertically holding arm and the horizontally pivoting arm 60 are in relatively high positions near the ceiling 36, the vertical arm 62 can be provided with the telescopic structure so that the display unit 14 can be positioned and held by operating the handle 104 to change the length of the vertical arm 62 between a low position in which the dental surgeon sits on the chair and treats the patient in the horizontal position and a high position in which the dental surgeon keeps standing and treats the patient in the standing position.

Further, by positioning the vertically holding arm 44 just above the patient 70 in the horizontal position or the standing position and holding the vertically holding arm 44 so as to direct the vertical axial line 72 of the vertically holding arm 44 to the oral cavity 70a of the patient 70, the display unit 14 can easily be moved around the vertical axial line 72 via the horizontally pivoting arm 60 and the vertically fixed arm 62, and held in an any position from eight o'clock to four o'clock around the patient.

Further, the empty space 74 enabling the dental surgeon to treat the patient 70 without interference with movement of the display unit 14 from eight o'clock to four o'clock from another member or equipment and, as depicted in FIG. 13, used to observe the oral cavity 70a of the patient 70 through the surgical microscope 10 positioned in the empty space can be secured above the patient 70.

It is to be noted that, though the telescopic structure of the vertically fixed arm 62 adopts manual operation with the handle 104, an actuator such as a motor may be provided so that the length of the vertically fixed arm 62 can be changed by remote control.

In addition, as a modified embodiment of the supporting mechanism depicted in FIG. 13 and FIG. 14, the horizontally pivoting arm 60 may be also provided with a stereoscopic structure in the same manner as the vertical arm 62 so that the length thereof can be changed. Further, as another modified embodiment of the supporting mechanism depicted in FIG. 13 and FIG. 14, the horizontally pivoting arm 60 and the vertical arm 62 may be an arm having a fixed length without being provided with the telescopic structure.

<Fourth Embodiment of Supporting Mechanism>

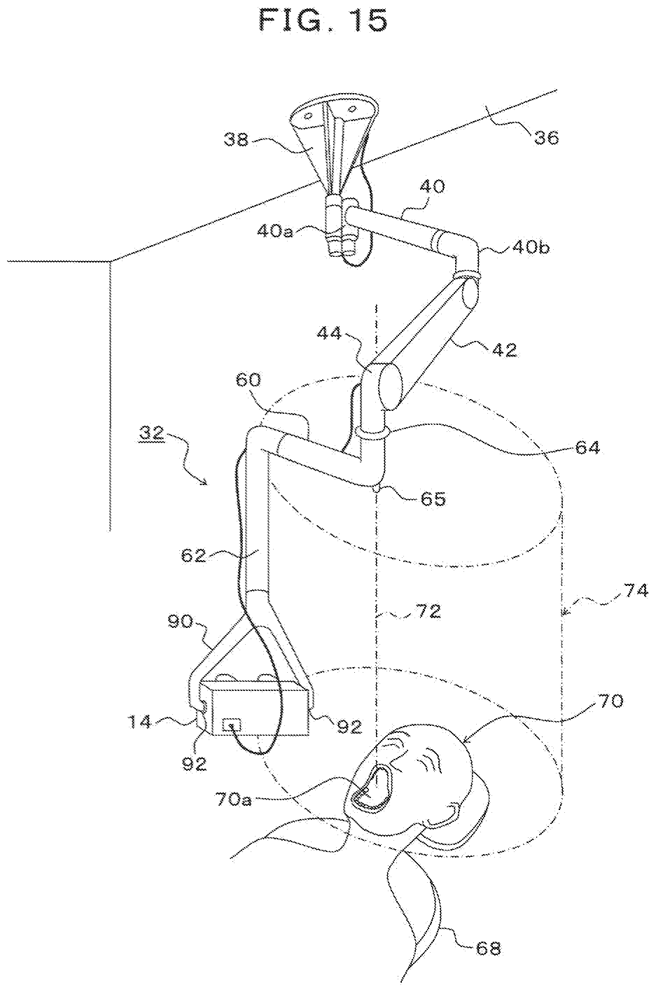

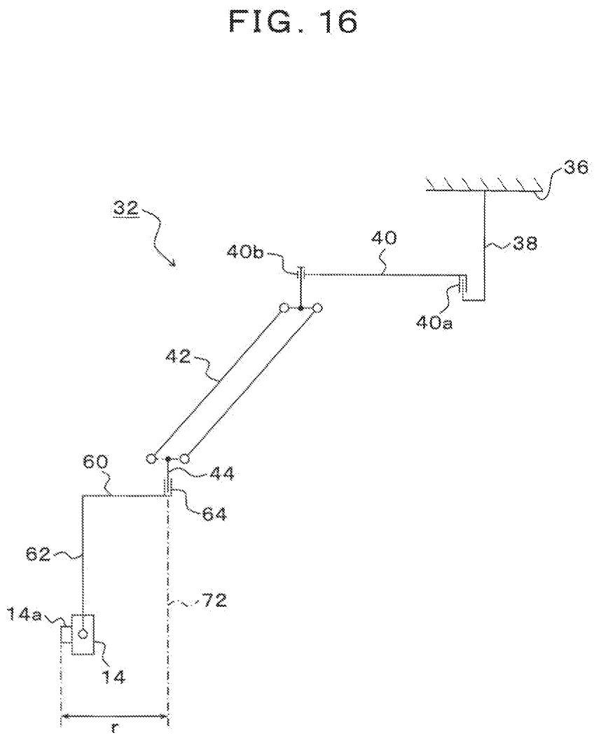

FIG. 15 is a descriptive diagram depicting a fourth embodiment of the supporting mechanism having a structure where the vertical arm supporting the display unit is fixed downwardly, and FIG. 16 is a descriptive diagram depicting a link configuration in the supporting mechanism depicted in FIG. 15.

As depicted in FIG. 15 and FIG. 16, the supporting mechanism 32 of the present embodiment is configured such that the vertical arm 62 is fixedly arranged downwardly at the distal end side of the horizontally pivoting arm 60. The other structures are the same as those of the first embodiment of the supporting mechanism 32 depicted in FIG. 3 and FIG. 4. It is to be noted that a right-angled arm 92 is not limited to being at aright angle to the horizontally pivoting arm 60 but may be extended outwardly and slightly obliquely.

Since the vertical arm 62 is fixedly arranged downwardly at the distal end side of the horizontally pivoting arm 60 in this manner, when the display unit 14 is moved pivotally on the axial line 72 of the vertically holding arm 44, the vertical arm 62 never moves vertically, so that the display unit can be horizontally pivoted smoothly without wobbling.

The structure where the vertical arm 62 is fixedly arranged downwardly at the distal end of the horizontally pivoting arm 60 in this manner can also be similarly applied to the supporting mechanism of the second embodiment depicted in FIG. 11 and FIG. 12 and the supporting mechanism of the third embodiment depicted in FIG. 13 and FIG. 14.

<Supporting Mechanism of any Dental Display>

The supporting mechanisms 32 depicted in FIT. 3 to FIG. 13 have been explained taking as an example the case of supporting the display unit 14 for stereoscopically viewing the observation image obtained by the surgical microscope so as to be movable three-dimensionally, but not only for the display unit for viewing the observation image taken by the surgical microscope, but also for all types of display units for displaying an image by any equipment or system for observing the teeth of a patient using an imaging device, the supporting mechanisms 32 depicted in FIG. 3 to FIG. 13 is used to move the display unit pivotally so as to depict a circle around the oral cavity of the patient so that the display unit can be easily and securely positioned in an optimum treatment position for the dental surgeon toward the face of the patient between eight o'clock and four o'clock.

Equipment or systems where the supporting mechanisms 32 depicted in FIG. 3 to FIG. 13 are used include an equipment, a system, and the like, for displaying on a display unit an observation image obtained by an imaging device provided at a distal end of an optical fiber scope.

<Modified Embodiment of the Present Invention>

The present invention is not limited to the embodiments described above but includes an observation system for medical treatment which can move a display unit for displaying an observation image for the medical treatment to a position optimum for the medical surgeon and hold the display unit.

In addition, the present invention is not limited to the above-described embodiments, but includes appropriate modifications which does not impair the object and advantages of the present invention, and further is not limited by numerical values shown in the above-described embodiments.

What is claimed is:

1. An observation system for dental and medical treatment, comprising:
a display unit for displaying a predetermined observation image regarding treatment of a patient on a dental chair; and
a supporting mechanism for supporting the display unit so as to be movable three-dimensionally and holding the display unit in an observation position of a dental surgeon keeping a constant posture, wherein
the supporting mechanism includes
a vertically holding arm supported so as to be movable to a position in which a vertical axial line passes through an oral cavity of the patient on the dental chair,
a horizontally pivoting arm having a rear end side supported on the vertically holding arm so as to be pivotable horizontally, and
a vertical arm provided downwardly at a distal end side of the horizontally pivoting arm, the display unit being attached to a distal end side of the vertical arm, and
the supporting mechanism has a structure where, in a state where the vertically holding arm has been moved to the position in which the vertical axial line of the vertically holding arm passes through an oral cavity of the patient on the dental chair, the display unit is supported so as to be movable for positioning via the vertical arm and the horizontally pivoting arm to a position around the dental chair corresponding to a predetermined pivoting radius defined by a horizontal distance up to a finder of the display unit around the vertical axial line.

2. The observation system for dental and medical treatment according to claim 1, wherein the vertical arm is arranged to a distal end side of the horizontally pivoting arm so as to be pivotable about a vertical line or be fixed downwardly.

3. The observation system for dental and medical treatment according to claim 1, wherein the display unit is supported on a distal end of the vertical arm so as to be turnable vertically.

4. The observation system for dental and medical treatment according to claim 1, wherein
between the base side and the vertically holding arm,
a second horizontally pivoting arm having a rear end side supported on the base side so as to be pivotable horizontally, and
a parallel link mechanism having a rear end side supported on a distal end side of the second horizontally pivoting arm so as to be pivotable horizontally for moving the vertically holding arm in three-dimensionally while keeping an attitude of the vertically holding arm provided on a distal end side of the parallel link mechanism are provided.

5. The observation system for dental and medical treatment according to claim 1, wherein, between the base side and the vertically holding arm, a parallel link mechanism having a rear end side supported on the base side so as to be pivotable horizontally for moving the vertically holding arm three-dimensionally while keeping a posture of the vertically holding arm provided on a distal end side of the parallel link mechanism is provided.

6. The observation system for dental and medical treatment according to claim 1, wherein the supporting mechanism is configured such that a rear end side of the vertically holding arm is supported on the base side so as to be pivotable horizontally.

7. The observation system for dental and medical treatment according to claim 1, wherein either one or both of the horizontally pivoting arm and the vertical arm are provided with a telescopic structure changing an arm length.

8. The observation system for dental and medical treatment according to claim 1, wherein the predetermined pivoting radius defined by the horizontal distance up to the finder of the display unit around the vertical axial line is set at a predetermined distance in a range from 10 cm to 30 cm.
